(19) United States
(12) Patent Application Publication
(10) Pub. No.: US 2026/0060614 A1
(43) Pub. Date: Mar. 5, 2026

(54) Systems and Methods for Dynamic Neurophysiological Stimulation

(71) Applicant: Cadwell Laboratories, Inc., Kennewick, WA (US)

(72) Inventors: Melissa Miles, West Linn, OR (US); Julia Aina Caviness, Collegeville, PA (US); Richard A. Villarreal, West Richland, WA (US); Ethan Rhodes, Benton City, WA (US)

(21) Appl. No.: 19/382,063

(22) Filed: Nov. 6, 2025

Related U.S. Application Data
(63) Continuation-in-part of application No. 18/638,809, filed on Apr. 18, 2024.
Application No. 18/638,809 is a continuation of application No. 16/402,544, filed on May 3, 2019, now Pat. No. 11,992,339.
(60) Provisional application No. 62/666,959, filed on May 4, 2018.

(51) Int. Cl. A61B 5/00; A61B 5/24; A61B 5/296; A61B 5/388; A61B 5/389; A61B 18/12

(52) U.S. Cl. CPC A61B 5/4893; A61B 5/24; A61B 5/296; A61B 5/388; A61B 5/389; A61B 5/4064; A61B 2018/1253; A61B 2018/126

(57) ABSTRACT

An intraoperative neurophysiological monitoring (IONM) system for identifying and assessing neural structures comprises at least one probe, at least one reference electrode, at least one strip or grid electrode, at least one sensing electrode, and a stimulation module. Threshold responses determined by stimulation during a surgical procedure are used to identify and assess functionality of neural structures. The identified neural structures are avoided and preserved while diseased or damaged tissue is resected during said surgical procedure.

### CROSS-REFERENCE

The present application is a continuation-in-part application of U.S. patent application Ser. No. 18/638,809, titled “Systems and Methods for Dynamic Neurophysiological Stimulation” and filed on Apr. 18, 2024, which is a continuation application of U.S. patent application Ser. No. 16/402,544, of the same title, filed on May 3, 2019, and issued as U.S. Pat. No. 11,992,339 on May 28, 2024, which, in turn, relies on U.S. Provisional Patent Application No. 62/666,959, titled “Systems and Methods for Neurophysiological Stimulation” and filed on May 4, 2018, for priority. All of the above referenced applications are hereby incorporated herein by reference in their entirety.

### FIELD

The present specification is related generally to the field of neurophysiological stimulation. More specifically the present specification is related to a stimulation module that enables user-preferred selection of one or any combination of a plurality of low current stimulation outputs or channels.

### BACKGROUND

Intraoperative neurophysiological monitoring (IONM) is directed towards identifying, mapping and monitoring neural structures in accordance with their functions with a goal of preserving the structural integrity of these neural structures during physically invasive procedures such as surgery.

In some methods, identifying, mapping and monitoring neural structures comprises applying electrical stimulation at or near an area where the target neural structures are believed to be located. Application of the electrical stimulation is transmitted through the nervous system structures to excite or depress the associated response(s) or function(s). For example, an electrical impulse is generated in the muscle(s), as a result of the excitation, that can be sensed using recording electrodes, thereby indicating presence and functionality of a neural structure to a surgeon. For example, cortical stimulation mapping (CSM) is a type of electrocorticography that involves a physically invasive procedure and aims to localize the function of specific brain regions through direct electrical stimulation of the cerebral cortex.

Prior art nerve integrity monitoring systems pose limitations when used across varied surgical procedures and accompanied neuro-stimulation scenarios. As an example, a critical limitation of majority of prior art nerve integrity monitoring systems is the availability of a limited number of low current outputs or channels for delivering stimulation to a plurality of neural regions thereby limiting the ability to simultaneously stimulate multiple nerves or multiple branches of single nerves. For example, the ES-IX stimulator, from Cadwell Industries Inc., also the Applicant of the present specification, has a maximum of one low current stimulation output. Such limitation necessitates frequent manual intervention, such as having to move the connections of stimulation components (for example, electrodes and probes) to change the location of the delivered stimulus on a patient's anatomy.

Another drawback of prior art nerve integrity monitoring systems is that these are not designed to provide electrical stimulation of sufficient amplitude to elicit excitation activity of the muscles. Another limitation is a lack of integration of the stimulators with a multi-modality monitoring system. For example, the Nicolet® Cortical Stimulator supports recording of only electroencephalography and connection of a single probe (bipolar). The Inomed® stimulator also supports connection of a single probe.

As a result of these limitations, prior art nerve integrity monitoring systems are associated with various disadvantages including the need for additional operational steps which increase the duration of the surgical procedures to the detriment of patients and medical personnel, increased complexity and confusion associated with intraoperative neural monitoring, a need for greater human and/or mechanical intervention and an inability to efficiently integrate multiple neural stimulation and monitoring modalities.

Thus, there is a need for systems and methods that enable a user to select all or any combination of multiple stimulation modalities available to the user. There is also a need to enable the user to stimulate the neurological system with minimal, less frequent and more streamlined manual or electromechanical intervention.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses an intraoperative neurophysiological monitoring (IONM) system for using cortical stimulation to assess neural structures during a surgical procedure, the system comprising: at least one reference electrode positioned in a perimeter of a surgical field of a patient; at least one probe; at least one strip electrode or grid electrode, wherein the at least one probe and/or the at least one strip electrode or grid electrode is positioned at target locations on the patient; at least one sensing electrode positioned on said patient's and configured to record said patient's responses to stimulation; a stimulation module, wherein the stimulation module is configured to: function in a first mode of operation or a second mode of operation depending upon a type of the at least one probe, wherein the first mode of operation is a bipolar mode and the second mode of operation is a monopolar mode; initiate a stimulation protocol in accordance with either the first mode of operation or second mode of operation; adjust stimulation parameters of the stimulation protocol to determine a threshold response; and use the at least one probe and/or the at least one strip electrode or grid electrode to determine whether the patient's neural structures are associated with at least one functional area based on the threshold response.

Optionally, the stimulation protocol is a motor cortex stimulation protocol, a speech stimulation protocol, or a language stimulation protocol.

Optionally, the stimulation module comprises a first plurality of output connectors and a second plurality of probe ports. Optionally, the first plurality of output connectors are configured to enable connection to the at least one strip electrode or grid electrode, wherein the at least one strip electrode or grid electrode comprises a plurality of contacts and wherein a total number of the plurality of contacts does not exceed a total number of the first plurality of output connectors. Optionally, the second plurality of probe ports comprises a first probe port and a second probe port, wherein each of the first probe port and the second probe port is configured to connect to the at least one probe, wherein the at least one probe comprises passive probes, and wherein the passive probes comprise at least one of a monopolar probe or a bipolar probe. Optionally, each of the first plurality of output connectors is configurable as either an anode or a cathode through a user interface in data communication with the IONM system. Optionally, each of the first plurality of output connectors is configurable as either an output for stimulation or an input for recording through a user interface in data communication with the IONM system. Optionally, the IONM system further comprises a computing device having a processor and a non-volatile memory for storing a plurality of programmatic instructions which, when executed, cause the processor to: provide a user interface in data communication with the IONM system; receive, via the user interface, user-defined stimuli, and deliver signals representative of the user-defined stimuli to pairs of the first plurality of output connectors, wherein each of the plurality of output connectors is configurable as either an anode or a cathode through the user interface. Optionally, a number of the first plurality of output connectors is equal to thirty-two.

Optionally, the at least one sensing electrode comprises an electromyography needle electrode.

Optionally, the stimulation protocol comprises a multi-pulse train having 2 to 10 pulses and wherein each of the pulses is defined by a pulse width in a range of 50 μsec to 1000 μsec, an inter-stimulus interval in a range of 0.5 to 10 milliseconds and a pulse amplitude in a range of 0.01 mA to 35 mA or 0.01V to 20V.

The present specification also discloses an intraoperative neurophysiological monitoring (IONM) system adapted to use direct nerve stimulation to identify nerve fibers and nerve pathways during a surgical procedure, the system comprising: at least one probe positioned at a first target location on the patient; at least one sensing electrode positioned at a second target location in the patient; and a stimulation module comprises a plurality of output connectors and a plurality of probe ports and is configured to: function in a first mode of operation or a second mode of operation depending upon a type of the at least one probe, wherein the first mode of operation is a bipolar mode and the second mode of operation is a monopolar mode, initiate a direct nerve stimulation protocol in accordance with either the first mode of operation or second mode of operation, adjust stimulation parameters of the direct nerve stimulation protocol to determine a threshold motor response, and use the at least one probe to identify certain of the patient's nerve fibers and nerve pathways based on the threshold motor response.

Optionally, the at least one strip electrode or grid electrode has a total number of contacts not exceeding a total number of the plurality of output connectors, wherein each of the plurality of output connectors is adapted to connect to the at least one strip electrode or grid electrode.

Optionally, the plurality of probe ports comprises a first probe port and a second probe port and is configured to connect to the at least one probe, wherein the at least one probe comprises at least one passive monopolar probe or passive bipolar probe.

Optionally, each of the plurality of output connectors is configurable as either an anode or a cathode.

Optionally, each of the plurality of output connectors is configurable as either an output for stimulation or an input for recording.

Optionally, the IONM system further comprises a computing device having a processor and a non-volatile memory for storing a plurality of programmatic instructions which, when executed, cause the processor to: provide a user interface in data communication with the IONM system; receive, via the user interface, user-defined stimuli, and deliver signals representative of the user-defined stimuli to pairs of the plurality of output connectors, wherein each of the plurality of output connectors is configurable as either an anode or a cathode through the user interface.

Optionally, the at least one sensing electrode comprises an electromyography needle electrode.

Optionally, the direct nerve stimulation protocol comprises a single pulse stimulation, wherein the single pulse has a frequency of 0.5 Hz to 130 Hz, a pulse width of 50 μsec to 1000 μsec, and an interval between pulses of 0.5 millisecond to 10 milliseconds. Optionally, a pulse amplitude is in a range of 0.01 mA to 35 mA or 0.01V to 20V for cranial nerves and in a range of 0.1 mA to 35 mA for peripheral nerves.

The present specification also discloses a method of using cortical stimulation to identify and assess neural structures during a surgical procedure, the method comprising: providing an intraoperative neurophysiological monitoring (IONM) system comprising at least one probe, at least one reference electrode, at least one strip electrode or grid electrode, at least one sensing electrode, and a stimulation module; placing the at least one reference electrode in a perimeter of a surgical field of a patient; positioning the at least one probe and/or the at least one strip electrode or grid electrode at target locations on the anatomy of said patient; preparing for recordation of said patient's responses to stimulation by positioning said at least one sensing electrode on said patient's anatomy; initiating a stimulation protocol; adjusting stimulation parameters of the stimulation protocol to determine a threshold response; and identifying or assessing the neural structures based on said threshold response, wherein assessing comprises determining if the patient's anatomy is functioning in a manner indicative of an underlying disease or, alternatively, is functioning in a non-pathological manner.

Optionally, the stimulation protocol is a motor cortex stimulation protocol, a speech stimulation protocol, or a language stimulation protocol.

Optionally, said stimulation module comprises a first plurality of output connectors and a second plurality of probe ports. Optionally, the first plurality of output connectors comprise at least 12 output connectors. Optionally, the first plurality of output connectors are configured to enable connection to the at least one strip electrode or grid electrode, wherein the at least one strip electrode or grid electrode has a plurality of contacts and wherein a total number of the plurality of contacts does not exceed a total number of the first plurality of output connectors. Optionally, the second plurality of probe ports comprises a first probe port and a second probe port, wherein each of the first probe port and the second probe port is configured to connect to the at least one probe, wherein the at least one probe comprises passive probes, and wherein the passive probes comprise at least one of a monopolar probe or a bipolar probe. Optionally, each of the plurality of output connectors are configurable as either an anode or a cathode through a user interface in data communication with the IONM system. Optionally, the method further comprises providing a user interface in data communication with the IONM system, receiving, via the user interface, user-defined stimuli, and delivering signals representative of the user-defined stimuli to pairs of the plurality of output connectors, each of the plurality of output connectors being configurable as either an anode or a cathode through the user interface. Optionally, the second plurality of probe ports comprises a probe port adapted to connect the at least one probe, wherein the at least one probe comprises an anode and a cathode and wherein the probe port comprises first and second outputs for connection of to the anode and the cathode of the at least one probe, a first pair of connection ports adapted to connect to a power supply and a second pair of connection ports adapted to connect to a communication module.

Optionally, the at least one sensing electrode comprises an electromyography needle electrode.

Optionally, the stimulation protocol comprises a multi-pulse train having 2 to 10 pulses wherein each of the pulses is defined by a pulse width in a range of 50 μsec to 1000 μsec, an inter-stimulus interval in a range of 0.5 to 10 milliseconds and a pulse amplitude in a range of 0.01 mA to 20 mA.

The present specification also discloses a method of using direct nerve stimulation to identify nerve fibers and nerve pathways during a surgical procedure, the method comprising: providing an intraoperative neurophysiological monitoring (IONM) system comprising at least one probe, at least one sensing electrode, and a stimulation module, wherein the stimulation module comprises at least twelve output connectors and a plurality of probe ports; positioning the at least one probe at a first target location on the patient; positioning the at least one sensing electrode at a second target location in the patient; initiating a direct nerve stimulation protocol; adjusting stimulation parameters of the direct nerve stimulation protocol to determine a threshold motor response; and identifying the nerve fibers and nerve pathways based on the threshold motor response.

Optionally, the IONM system further comprises at least one strip electrode or grid electrode having a total number of contacts not exceeding a total number of the at least twelve output connectors, wherein the at least twelve output connectors are adapted to connect to the at least one strip electrode or grid electrode.

Optionally, the plurality of probe ports comprises a first probe port and a second probe port and is configured to connect to the at least one probe, wherein the at least one probe comprises at least one passive monopolar probe or passive bipolar probe.

Optionally, each of the at least twelve output connectors are configurable as either an anode or a cathode.

Optionally, the method further comprises providing a user interface in data communication with the IONM system, receiving, via the user interface, user-defined stimuli, and delivering signals representative of the user-defined stimuli to pairs of the plurality of output connectors, each of the plurality of output connectors being configurable as either an anode or a cathode through the user interface.

Optionally, the plurality of probe ports comprises a probe port configured to connect to the at least one probe, wherein the at least one probe comprises an anode connection and a cathode connection, and wherein the probe port comprises first and second outputs adapted to connect to the anode and the cathode of the probe port, a first pair of connection ports adapted to connect to a power supply and a second pair of connection ports adapted to connect to a transceiver.

Optionally, the at least one sensing electrode comprises an electromyography needle electrode.

Optionally, the direct nerve stimulation protocol comprises a single pulse stimulation, wherein the single pulse has a frequency of 0.05 Hz to 90 Hz, a pulse width of 50 μsec to 1000 μsec, an interval between pulses of 0.5 millisecond to 10 milliseconds and a pulse amplitude in a range of 0.01 mA to 20 mA, with 2 mA or less for cranial nerves and 5 mA or less for peripheral nerves.

Optionally, the IONM system further comprises a handle having a proximal end configured to connect to the stimulation module and a distal end configured to attach to the at least one probe. Optionally, the handle comprises a first visual indicator, a second visual indicator, and an actuator configured to manually or automatically switch the stimulation module between a first mode of operation and a second mode operation depending upon a type of the at least one probe. Optionally, first visual indicator is configured to indicate at least one of the first mode of operation, the second mode of operation, a connection state of the at least one probe or what part of the at least one probe is active. Optionally, the second visual indicator provides a first indication signifying that a site of stimulation is at a first distance from a nerve and a second indication signifying that the site of stimulation is at a second distance from the nerve, wherein the first distance is less than the second distance.

The present specification also discloses a method of using cortical stimulation to identify neural structures during a surgical procedure, the method comprising: providing an intraoperative neurophysiological monitoring (IONM) system comprising at least one probe, at least one reference electrode, at least one strip or grid electrode, at least one sensing electrode, and a stimulation module; placing said at least one reference electrode in a perimeter of a surgical field of a patient; positioning said at least one probe and said at least ones strip or grid electrode at target locations on the anatomy of said patient; preparing for recordation of said patient's responses to stimulation by positioning said at least one sensing electrode on said patient's anatomy; initiating a stimulation protocol; adjusting stimulation parameters of said stimulation protocol to determine a threshold response; and identifying said neural structures based on said threshold response.

Optionally, the stimulation protocol is a motor cortex stimulation protocol, a speech stimulation protocol, or a language stimulation protocol.

Optionally, said stimulation module comprises a first plurality of twelve output connectors and a second plurality of probe ports.

Optionally, said twelve output connectors enable connection to said at least one strip or grid electrode having multiple contacts not exceeding said twelve output connectors.

Optionally, said plurality of probe ports comprises a first probe port and a second probe port and enables connection to said at least one probe, wherein said at least one probe comprises passive and smart probes, and wherein said passive probes include monopolar and bipolar probes.

Optionally, each of the twelve output connectors are configurable as either an anode or a cathode allowing user-defined stimuli to be delivered to arbitrary anode and cathode pairs.

Optionally, said second plurality of probe ports further comprises a third probe port, said third probe port configured for connecting a smart probe and comprising a pair of outputs for connection of an anode and a cathode of said smart probe and a first connection port for power and a second connection port for communications.

Optionally, said at least one sensing electrode includes an EMG needle electrode.

Optionally, said stimulation protocol comprises a multi-pulse train of 3 to 5 pulses, a pulse width of 500 μsec, an inter-stimulus interval of 2 to 4 milliseconds and a pulse amplitude of up to 20 mA.

The present specification also discloses a method of using direct nerve stimulation to identify nerve fibers and pathways during a surgical procedure, the method comprising: providing an intraoperative neurophysiological monitoring (IONM) system comprising at least one probe, at least one sensing electrode, and a stimulation module; positioning said at least one probe at a target location on the anatomy of said patient; preparing for recordation of said patient's responses to stimulation by positioning said at least one sensing electrode on said patient's anatomy; initiating a direct nerve stimulation protocol; adjusting stimulation parameters of said stimulation protocol to determine a threshold motor response; and identifying said nerve fibers and pathways based on said threshold motor response.

Optionally, said stimulation module comprises a first plurality of twelve output connectors and a second plurality of probe ports.

Optionally, said twelve output connectors enable connection to at least one strip or grid electrode having multiple contacts not exceeding said twelve output connectors.

Optionally, said plurality of probe ports comprises a first probe port and a second probe port and enables connection to said at least one probe, wherein said at least one probe comprises passive and smart probes, and wherein said passive probes include monopolar and bipolar probes.

Optionally, each of the twelve output connectors are configurable as either an anode or a cathode allowing user-defined stimuli to be delivered to arbitrary anode and cathode pairs.

Optionally, said second plurality of probe ports further comprises a third probe port, said third probe port configured for connecting a smart probe and comprising a pair of outputs for connection of an anode and a cathode of said smart probe and a first connection port for power and a second connection port for communications.

Optionally, said at least one sensing electrode includes an EMG needle electrode.

Optionally, said direct nerve stimulation protocol comprises single pulse stimulation, wherein each stimulation has a frequency of 2 to 3 Hz, a pulse width of 200 μsec, an inter-stimulus interval of 1 millisecond and a pulse amplitude of 0.01 mA to 20 mA, with 2 mA or less for cranial nerves and 5 mA or less for peripheral nerves.

The aforementioned and other embodiments of the present shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a block diagram illustration of an Intraoperative Neuro-Monitoring (IONM) system, in accordance with an embodiment of the present specification;

FIG. 1B illustrates a multi-modality stimulation module, in accordance with an embodiment of the present specification;

FIG. 1C illustrates a handle in communication with the multi-modality stimulation module of FIG. 1B through an electrical connector, in accordance with an embodiment of the present specification;

FIG. 1D illustrates a multi-modality stimulation module in accordance with other embodiments of the present specification;

FIG. 2 illustrates a monopolar probe, in accordance with an embodiment of the present specification;

FIG. 3 illustrates a bipolar probe, in accordance with an embodiment of the present specification;

FIG. 4A illustrates a strip electrode configured in a monopolar setup, in accordance with an embodiment of the present specification;

FIG. 4B illustrates the strip electrode of FIG. 4A configured in a bipolar setup, in accordance with an embodiment of the present specification;

FIG. 5 shows a tumor or epileptogenic region with reference to a map of the functional areas of a human brain, in accordance with an embodiment of the present specification;

FIG. 6 is a flowchart illustrating a plurality of steps of a use case of cortical stimulation, using the IONM system of the present specification; and,

FIG. 7 is a flowchart illustrating a plurality of steps of another use case of direct nerve stimulation, using the IONM system of the present specification.

### DETAILED DESCRIPTION

A “computing device” is at least one of a cellular phone, PDA, smart phone, tablet computing device, patient monitor, custom kiosk, or other computing device capable of executing programmatic instructions. It should further be appreciated that each device and monitoring system may have wireless and wired receivers and transmitters capable of sending and transmitting data. Each “computing device” may be coupled to at least one display, which displays information about the patient parameters and the functioning of the system, by means of a GUI. The GUI also presents various menus that allow users to configure settings according to their requirements. The system further comprises at least one processor (not shown) to control the operation of the entire system and its components. It should further be appreciated that the at least one processor is capable of processing programmatic instructions, has a memory capable of storing programmatic instructions, and employs software comprised of a plurality of programmatic instructions for performing the processes described herein. In one embodiment, the at least one processor is a computing device capable of receiving, executing, and transmitting a plurality of programmatic instructions stored on a volatile or non-volatile computer readable medium. In addition, the software comprised of a plurality of programmatic instructions for performing the processes described herein may be implemented by a computer processor capable of processing programmatic instructions and a memory capable of storing programmatic instructions.

The term ‘user’ is used interchangeably to refer to a surgeon, neuro-physician, neuro-surgeon, neuro-physiologist, technician or operator of the IONM system and/or other patient-care personnel or staff.

The term ‘passive probe’ refers to a monopolar or bipolar probe that does not include active electronic components (such as amplifiers, signal conditioning circuits, or digital control elements) within its body and instead relies entirely on an external stimulation module or amplifier (for example, multi-modality stimulation module 120 and multi-modality stimulation module 190) to generate, control, and monitor electrical signals. In contrast, an ‘active probe’ or ‘smart probe’ incorporates embedded electronic circuitry (such as, for example, microcontrollers, digital identifiers, or solid-state switching elements) that allow it to alter its configuration or functionality dynamically under IONM software control. For example, a smart probe may electronically switch between monopolar and bipolar modes or communicate its configuration to the IONM system via a digital interface.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

### An Intraoperative Neuro-Monitoring (IONM) System

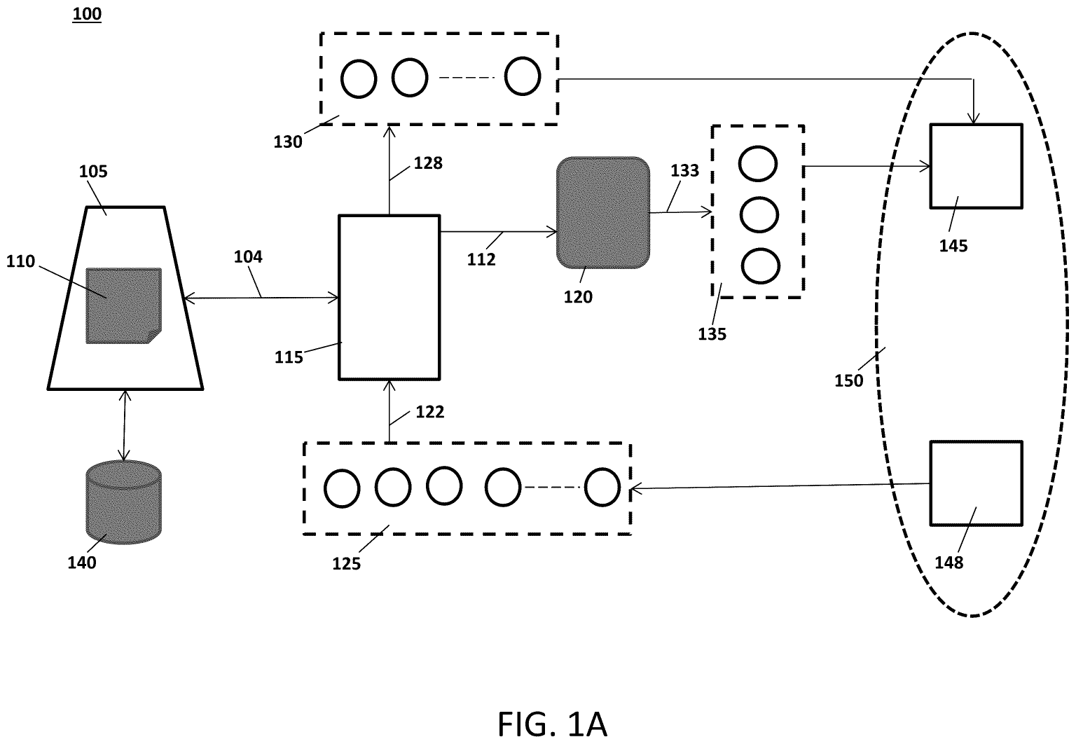

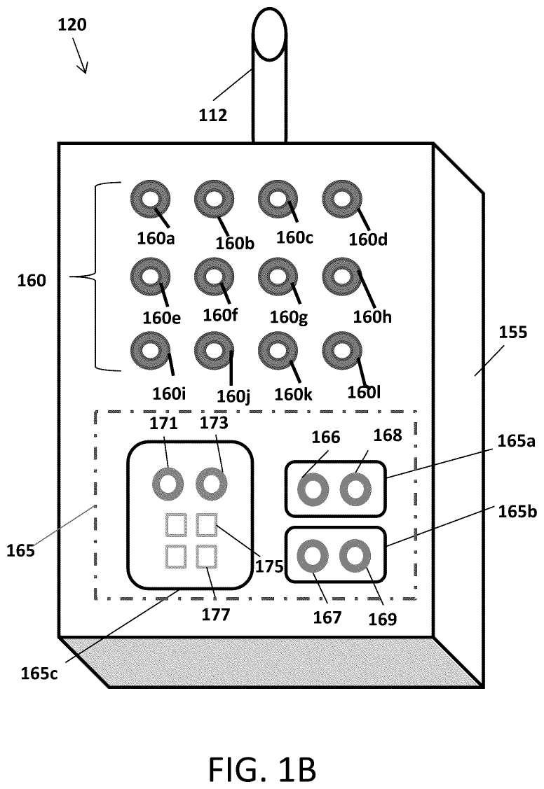

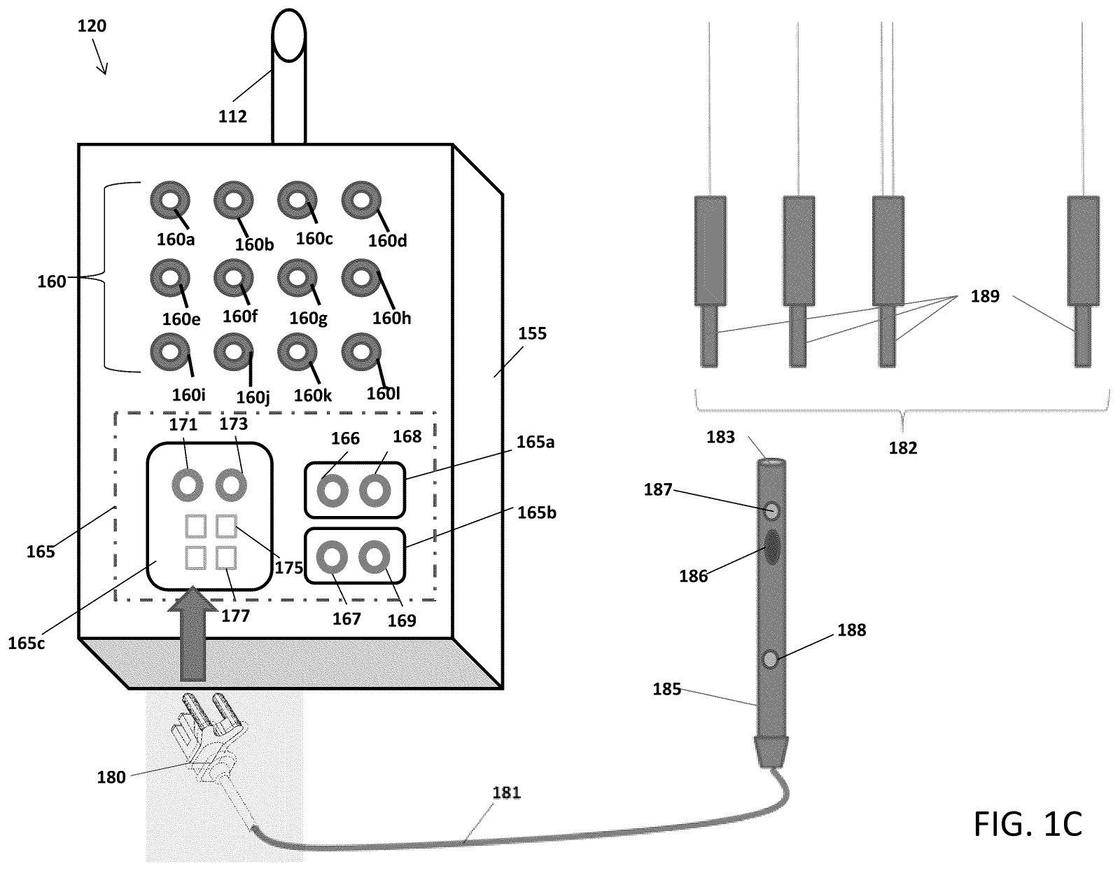

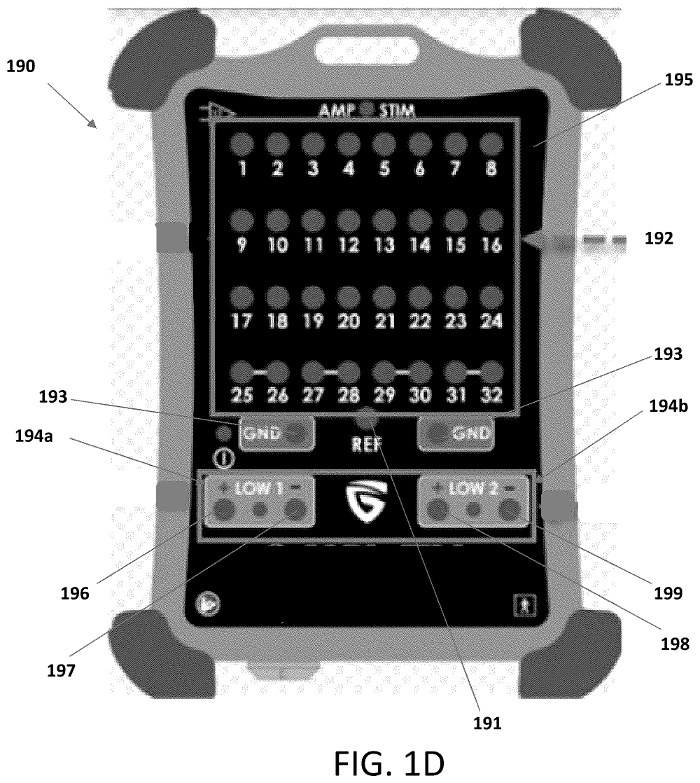

FIG. 1A is a block diagram illustration of an IONM system 100, in accordance with an embodiment of the present specification. In embodiments, the system 100 enables stimulation based assessment of nerve proximity, direction, pathways and/or changes to nerve pathology, health or status during physically invasive procedures. The system 100 comprises a computing device 105 capable of implementing or executing an IONM software application or engine 110, at least one multi-connection console 115 connected to the computing device 105 using a cable 104, a multi-modality stimulation module 120 connected to the console 115 using a cable 112, a plurality of stimulation components 135 such as, but not limited to, a monopolar probe, a bipolar probe, and a strip or grid electrode along with an integrated or discrete reference electrode capable of being coupled to the stimulation module 120 simultaneously or in any combination thereof via respective cables 133, a plurality of recording or sensing electrodes such as, but not limited to, EMG (Electromyography) electrodes 125 connected to the console 115 through respective cables 122 and a plurality of surgical instruments, components and accessories 130 coupled to the console 115 via respective accessory cables 128. In embodiments of the present specification, the multi-modality stimulation module 120 comprises a plurality of connection ports such that more than one stimulation component 135 may be connected to the system 100 at the same time. Specifically, both a monopolar probe and a bipolar probe can be simultaneously attached to the multi-modality stimulation module 120 without having to unplug and re-plug the probes to switch back and forth between the probes.

In various embodiments, the computing device 105 comprises at least one processor, at least one non-transitory memory, one or more input devices (such as, but not limited to, keyboard, mouse, touch-screen, camera and combinations thereof) and one or more output devices (such as, but not limited to, display screens, printers, speakers and combinations thereof) all of which may be stand-alone, integrated into a single unit, partially or completely network-based or cloud-based, and not necessarily located in a single physical location. The computing device 105 is in data communication with one or more databases 140 that may be co-located with the computing device 105 or located remotely.

The IONM software application or engine 110 implements a plurality of instructions to: deliver a plurality of stimulation protocols or schedules (stored in the one or more databases 140) through any one, any combination or all of the plurality of stimulation components 135, generate a plurality of graphical user interfaces (GUIs) rendered on one or more display screens (that are coupled to the computing device 105) to display a plurality of EMG activity waveforms sensed by the EMG electrodes 125 and extract a plurality of parameters related thereto and enable user-interaction with the system 100 to perform a plurality of functions such as, but not limited to, selecting and activating/initiating one or more stimulation protocols and modulating one or more stimulation parameters of the protocols. The IONM software application or engine 110 is configured to apply one or more stimulation protocols to one or more nerve structures 145 of a patient 150 through the plurality of stimulation components 135 and acquire and record correspondingly evoked EMG activity through the plurality of EMG electrodes 125 positioned within a plurality of muscle sites or locations 148 of the patient 150.

The systems and methods of the embodiments of the present specification are used for mapping and locating anatomical structures and also for assessing these structures, wherein assessing is defined as determining if these structures are functioning in a manner indicative of an underlying disease or, alternatively, are functioning in a non-pathological manner. The functions include, but are not limited to, cognitive functions such as speech and language and motor functions (movement). A neural structure is determined to be functioning based on the presence or absence of a non-pathological response when stimulated. In some embodiments, for a motor response, a non-pathological response is defined as movement or non-movement of a muscle group. In some embodiments, for a cognitive response, a non-pathological response is defined as a patient properly reading a sentence aloud correctly naming a pictured object. It should be appreciated by those of ordinary skill in the art that, although described herein with reference to cortical stimulation and direct nerve stimulation during cerebrospinal surgical procedures, the system 100 and related methods or use cases of the present specification have application in a plurality of surgical procedures during which tissue having critical neural structures must be approached, retracted, and/or impinged upon and consequently requiring that such physically invasive procedures be planned and executed while preserving critical neural structures or bundles. It should also be appreciated that, although embodiments have been described herein with reference to EMG activity, the system 100 and related methods or use cases of the present specification may, in various alternate embodiments, use a plurality of different types of neural monitoring modalities such as, for example, triggered electromyography, spontaneous electromyography, mechanomyography, somatosensory evoked potential, motor evoked potentials, nerve conduction velocity and/or train of fours.

### The Multi-Modality Stimulation Module

FIG. 1B illustrates a multi-modality stimulation module 120, in accordance with an embodiment of the present specification. Referring to FIGS. 1A and 1B, the module 120 comprises a housing or enclosure 155 connected, in some embodiments, to a distal end of the electrical cable 112 while a proximal end of the cable 112 is connected to the console 115. In alternate embodiments, the proximal end of the cable 112 may be connected directly to the computing device 105 via a connector such as a D-subminiature connector. In other alternate embodiments, the module 120 may be connected to the console 115 through the electrical cable 112 that serves only to deliver power to the module 120, while the module 120 is in wireless data communication with the computing device 105. Also, in some embodiments, the module 120 is configured as a hand-held device.

In some embodiments, the module 120 comprises a first plurality of connectors 160 and a second plurality of separate probe ports 165 (that is, ports 165a, 165b and 165c). In accordance with an embodiment, the first plurality of connectors 160 comprises twelve anode/cathode ports or connectors (160a, 160b, 160c, 160d, 160e, 160f, 160g, 160h, 160i, 160j, 160k, 160l) while the second plurality of separate probe ports 165 comprises a first probe port 165a, a second probe port 165b, and a third probe port 165c.

In embodiments, the first probe port 165a and the second probe port 165b are used to connect either a monopolar probe or a bipolar probe and, in some embodiments, the third probe port 165c is used to connect a smart probe. The first probe port 165a comprises a first output 166 and a second output 168 for connection of an anode and a cathode of a first probe. The second probe port 165b comprises a third output 167 and a fourth output 169 for connection of an anode and a cathode of a second probe. In some embodiments, the third probe port 165c comprises a fifth output 171 and a sixth output 173 for connection of an anode and a cathode of a smart probe and a first pair of connection port 175 for power and a second pair of connection port 177 for communications for the smart probe.

The first probe port 165a and the second probe port 165b are both configured to receive either a monopolar probe or a bipolar probe. Therefore, the first probe port 165a and the second probe port 165b allow both a monopolar probe and a bipolar probe to be simultaneously attached to the multi-modality stimulation module 120. A user may perform a procedure on a patient without having to unplug and re-plug monopolar and bipolar probes to switch back and forth between the probes. In some embodiments, the system 100 includes a switching circuit configured to switch each of the twelve ports or connectors 160a-1 and each of the outputs 166, 167, 168, 169, 171, 173 such that each connector 160a-l or each output 166, 167, 168, 169, 171, 173 can function as either a cathode output or an anode output.

In embodiments, the twelve ports 160a-1601 enable connection to one or more strips, each of which has multiple contacts, not exceeding the twelve ports or channels. In accordance with an embodiment, any of the twelve ports 160a-1601 can be configured and flexibly chosen as either an anode or a cathode, thereby allowing user-defined stimuli to be delivered to arbitrary anode and cathode pairs. In one embodiment, a strip or grid electrode, which includes a reference electrode as part of the collection of electrodes contained therein, is connected to the required number of output ports from the available twelve ports 160a-1601.

In some embodiments, the second plurality of separate probe ports 165 (that is, ports 165a, 165b and 165c) enables connection to passive and smart probes. In various embodiments, the passive stimulation probes are monopolar and bipolar probes. The smart probe dynamically switches between stimulation functions and provides visual and/or auditory feedback to the user about one or more characteristics (such as, but not limited to, amplitude, latency, location of response, similarity to prior and/or baseline response) of a sensed/detected response. The smart probe (that is available in either a monopolar or a bipolar version) enables the user to control stimulation parameters whereas the passive monopolar and bipolar probes require dependency on another user to adjust parameters using the IONM software engine 110.

In some embodiments, as shown in FIG. 1C, an electrical connector 180 is configured to engage (and disengage) with the third probe port 165c comprising the outputs 171, 173 and the connection port pairs 175, 177. A proximal end of a handle 185 is connected to the connector 180 through a cable 181. A distal end of the handle 185 is configured to (detachably) receive, hold or support a plurality of types (monopolar and bipolar) and sub-types of probe tips 182 such as, for example, a monopolar probe, a monopolar ball tip probe, a bipolar probe, a monopolar/bipolar concentric probe, a monopolar/microfork probe or a monopolar/bipolar prong probe. In embodiments, the probe tips 182 have a common base or connector 189 configured to connect to the handle 185 via a receiving port 183 of the handle. Thus, a plurality of different probe tips can be used with the single handle 185 that is connected to the multi-modality stimulation module 120 via the connector 180. In some embodiments, a design of the electrical connector 180 is of the design disclosed in the assignee's application Ser. No. 29/378,861, now U.S. Pat. No. D670656, which is hereby incorporated by reference. In embodiments, the handle 185 and probe tip 182 comprise a ‘smart probe’ and are configured to connect to the module 120 via the connector 180, cable 181, and third probe port 165c.

In some embodiments, the handle 185 has an actuator 186, such as a toggle button, to enable manual switching between monopolar and bipolar modes depending upon the type or subtype of probe tip attached to the handle 185 or a sensing switch configured to enable an automatic switching between modes depending upon the type or subtype of probe tip attached to the handle 185. In some embodiments, the handle 185 also has a visual (light) indicator 187 that indicates monopolar or bipolar modes depending upon the type of probe tip 182 attached to the handle 185, an active or inactive connection state or status of the probe tip 182 attached to the handle 185 and/or which part (monopolar or bipolar) of the prong probe type is active when a monopolar/bipolar prong probe is attached to the handle 185. In some embodiments, the handle 185 further has a visual (light) proximity indicator 188 to provide visual feedback indicative of whether a nerve is far or near from a site where stimulation is being applied. It should be appreciated that the indicator 188 eliminates the need for the user to repeatedly look at a display screen of the IONM system 100. In some embodiments, the proximity indicator 188 is configured to generate or provide at least two indications-a first visual indication (such as, for example, green) signifying that a high stimulation intensity (that is, a stimulation intensity above a predefined threshold stimulation intensity) is required to elicit an evoked response thereby meaning that the site of stimulation is ‘far’ from the nerve, and a second visual indication (such as, for example, red) signifying that a relatively low stimulation intensity (that is, a stimulation intensity below a predefined threshold stimulation intensity) is required to elicit an evoked response thereby meaning that the site of stimulation is ‘near’ or ‘close’ to the nerve.

In embodiments, the power port pair 175 provides power to the handle 185 and probe tip 182 through the connector 180 and cable 181. In embodiments, the communication port pair 177 enables the proximity indicator 188, recordation of the type of stimulator being used and a type of mode (i.e. monopolar and/or bipolar) in clinical data of the IONM system 100, and connection state (i.e. connected or disconnected) of the handle 185 and probe tip 182 type, via the connector 180 and cable 181. The communication port pair 177 may be in data communication with a transceiver to enable the transfer of data.

FIG. 1D illustrates another embodiment of a multi-modality stimulation module 190 featuring a port architecture that extends functional flexibility and clinical utility. Specifically, multi-modality stimulation module 190 includes a plurality of advanced multi-purpose ports 192, each of which can be independently configured as an anode, a cathode, or a recording input based on procedural needs.

In some embodiments, the multi-modality stimulation module 190 integrates a stimulator with a 32-channel amplifier to support coordinated stimulation and recording within the same system architecture. In embodiments, the stimulator is configured to generate timed, controlled electrical pulses used to excite one or more neural structures in order to evoke a measurable physiological response. The stimulator is configured to generate low-current electrical pulses for direct stimulation of cortical and subcortical areas. Stimulation may be delivered either through a dedicated probe or through the same electrodes used for recording. The amplifier is configured such that it can temporarily disable recording for the active stimulating electrodes only when the same electrodes are used for both stimulation and recording, thereby preventing signal saturation or artifact contamination during the stimulation event. Immediately following the stimulation pulse, the corresponding amplifier inputs are enabled again (“opened”) to capture the evoked electrophysiological responses, such as EEG or ECOG (electrocorticography) activity.

In some embodiments, the stimulator and the integrated 32-channel amplifier are internally synchronized to ensure that a) the amplifier uses a blanking time window during generation of stimulation pulses by the stimulator and immediately after the stimulation pulses (to avoid amplifier saturation from stimulus artifacts), and b) the amplifier opens an acquisition time window to acquire post-stimulation evoked EMG or EEG signals. Stated differently, in some embodiments, the amplifier and stimulator are time-synchronized, enabling precise coordination of stimulation delivery and data acquisition. The synchronization of stimulation triggers and acquisition sweeps is managed by a host software, in some embodiments, rather than through hardware-based timing circuits. This software-based synchronization allows flexible scheduling and adjustment of stimulation and recording sequences, ensuring that the amplifier operates with appropriate blanking intervals during stimulation and controlled acquisition windows for post-stimulation response capture.

The amplifier is configured to amplify, filter and digitize the acquired evoked EMG or EEG signals. In some embodiments, the stimulator may be configured to send a trigger pulse to the amplifier to initiate acquisition of post-stimulation evoked EMG or EEG activity.

In some embodiments, as shown in FIG. 1D, the multi-modality stimulation module 190 includes a first plurality of connectors 192 comprising thirty-two ports or connectors (labelled 1-32). Further, the multi-modality stimulation module 190 includes a first plurality of probe ports 194a, comprising probe ports 196, 197, and a second plurality of probe ports 194b, comprising probe ports 198, 199).

In some embodiments, the systems of the present specification include a switching circuit configured to switch each of the thirty-two ports or connectors 192, such that a) any of the thirty-two ports or connectors 192 is configurable as anode or cathode, and b) any of the thirty-two ports or connectors 192 is configurable as an output for stimulation or input for recording. Thus, the stimulator 190 of FIG. 1D comprises thirty-two ports or connectors (each configurable as input or output and further configurable as anode or cathode) compared to the twelve ports or connectors (each functioning only as an output and further configurable as anode or cathode) in the stimulator 120 of FIG. 1B. Further, in some embodiments, the switching circuit is configured to switch any of the probe outputs 196, 197, 198 and 199 as either a cathode output or an anode output. That is, the anode/cathode polarity can be set to normal or reverse and switched as needed using software-based controls, allowing dynamic reconfiguration during operation.

In some embodiments, the switching circuit includes a switch matrix comprised of three high-voltage isolated switches per I/O pin-namely, an amplifier-patient switch, a stimulation output switch, and a stimulation return switch. The control lines governing these switches are electrically isolated from all patient connections to maintain safety and signal integrity. These control lines are driven through serial-to-parallel shift registers arranged in a daisy-chain configuration, such that a single communication bus can control up to ninety-six switches efficiently. Additionally, the system is configured such that switching sequences can be scheduled intelligently to minimize artifacts or transients that might otherwise appear in the acquired physiological data. This architecture enables precise, software-defined control of stimulation and recording pathways while maintaining high patient safety and signal fidelity.

In embodiments, the thirty-two ports 192 enable connection to one or more strips or grid electrodes, each of which has multiple contacts, not exceeding the thirty-two ports or channels in total. In accordance with an embodiment, any of the thirty-two ports 192 can be configured and flexibly chosen as either an anode or a cathode, thereby allowing user-defined stimuli to be delivered to arbitrary anode and cathode pairs. Further, any of the thirty-two ports can be configured and flexibly chosen as either an output for stimulation or input for recording. In one embodiment, a strip or grid electrode, which includes a reference electrode (which may be connected to port 191) as part of the collection of electrodes contained therein, is connected to the required number of output ports from the available thirty-two ports. The multi-modality stimulation module 190 also comprises two ground ports 193.

In embodiments, each contact on a strip or grid electrode is configured to terminate into an individual touch-proof connector, thereby maintaining a one-to-one correspondence between electrode contacts and the available input/output ports of the stimulation module 190. For instance, a 1×4 grid electrode having four contacts terminates into four separate connectors, each of which is connected to a distinct port among the thirty-two available ports 192. This configuration allows each contact on the strip or grid to be individually handled, addressed, and/or controlled via the associated software. Consequently, each contact can be selectively and flexibly configured as an anode, cathode, output for stimulation or recording input, depending on the intended stimulation or monitoring mode. Users may thereby define customized stimulation or recording channel configurations, from the four contacts, such as referencing adjacent contacts (e.g., grid contact 1 to grid contact 2, grid contact 2 to grid contact 3, and so on) or referencing each grid contact to an independent electrode, such as a cranial reference (e.g., grid contact 1 to Cz, grid contact 2 to Cz, and so forth). This architecture enables precise, software-defined control of individual electrode contacts within multi-contact grid or strip arrays.

Referring to FIGS. 1A and 1D, the multi-modality stimulation module 190 comprises a housing or enclosure 195 connected, in some embodiments, to a distal end of the electrical cable 112 while a proximal end of the cable 112 is connected to the console 115. In some embodiments, the cable 112 is a keyed circular LEMO connector. In alternate embodiments, the proximal end of the cable 112 may be connected directly to the computing device 105 via a connector such as a USB connector. It should be appreciated that while the stimulation module 120 provides stimulation only and requires a separate amplifier for recording, the multi-modality stimulation module 190 provides stimulation and recording in the same device.

In embodiments, the first plurality of probe ports 194a and the second plurality of probe ports 194b are used to connect either a passive monopolar probe or a passive bipolar probe. The first plurality of probe ports 194a comprises a first output 196 and a second output 197 for connection of an anode and a cathode of a first probe. The second plurality of probe ports 194b comprises a third output 198 and a fourth output 199 for connection of an anode and a cathode of a second probe. The first plurality of probe ports 194a and the second plurality of probe ports 194b are configured to allow both a passive monopolar probe and a passive bipolar probe to be simultaneously connected to the multi-modality stimulation module 190. Each port of the plurality of probe ports 194a, 194b can be independently configured to operate in a desired mode, such as stimulation or recording. However, in some embodiments, while both the plurality of probe ports 194a and the plurality of probe ports 194b may be active and configured concurrently, simultaneous or overlapping stimulation events across the probe ports is not supported. A user may perform a procedure on a patient without having to unplug and re-plug monopolar and bipolar probes to switch back and forth between the probes.

In some embodiments, the IONM software engine 110 is configured to automatically detect whether a passive monopolar probe or a passive bipolar probe is connected by the user and, based on that detection, to automatically configure the multi-modality stimulation module 190 to operate in the corresponding monopolar or bipolar stimulation mode. The automatic detection is enabled, for example, by monitoring probe connection patterns, impedance characteristics, or specific connector pin configurations associated with each probe type. Alternatively, the system may allow the user to manually input or select the desired configuration through a user interface generated by the IONM software engine 110, thereby instructing the multi-modality stimulation module 190 to function in either monopolar or bipolar mode.

Configuring the multi-modality stimulation module 190 to operate in monopolar or bipolar stimulation mode refers to how the electrical return path for current flow during stimulation is defined—that is, how the anode and cathode are assigned among the connected electrodes. In monopolar stimulation mode, a single active electrode (the cathode) delivers a stimulation current directly to a remote reference or return electrode (the anode), which is typically placed at a distant, electrically neutral site (such as, for example, the scalp, shoulder, or other body location). The current therefore flows between a localized stimulating electrode and a distant return site, producing an asymmetric electric field that can activate a relatively larger volume of neural tissue. Consequently, monopolar stimulation mode is often used when a wider or deeper field of excitation is desired, such as during motor mapping or subcortical stimulation. In contrast, in bipolar stimulation mode, both the cathode and anode are located on adjacent contacts of the same probe. The stimulation current flows locally between these two nearby contacts, generating a more confined and focused electric field. Bipolar stimulation therefore provides spatially restricted activation, reducing current spread and minimizing stimulation of surrounding or distant neural structures. It is typically preferred for precise cortical mapping or when localized stimulation control is required.

In some embodiments, the first plurality of probe ports 194a and the second plurality of probe ports 194b enable connection to passive and active or smart probes. In various embodiments, the passive stimulation probes are monopolar and bipolar probes. The smart probe changes its functional mode between monopolar and bipolar stimulation operations in real time, under the control of the IONM software engine 110 or a user (such as a surgeon user), without requiring physical disconnection, manual rewiring, or replacement of the probe. This change in functional mode can occur repeatedly during a procedure. In stimulation mode, the probe delivers electrical pulses to one or more neural structures.

In some embodiments, the change in functional mode may be triggered by: a) user input through a button or a touch sensor integrated into a handle of the smart probe, and/or b) command issued from the IONM software engine 110 based on, for example, a pre-programmed sequence.

The smart probe comprises programmable electrode contact(s) and embedded electronics (for example, a microcontroller or an ASIC (Application-Specific Integrated Circuit)) that receives the functional mode-selection commands and accordingly permits dynamic configuration of the smart probe as stimulation output or recording input. In some embodiments, the embedded electronics interfaces with a bi-directional analog switch capable of routing the electrode contact(s) to either a stimulator output circuitry or an amplifier input path.

In some embodiments, the smart probe provides real-time visual and/or auditory feedback to the user through one or more LEDs (Light Emitting Diodes) and/or audio elements, conveying one or more characteristics of detected physiological responses such as, but not limited to, amplitude, latency, and similarity to baseline. In some embodiments, the one or more LEDs may be located near a distal tip or handle of the smart probe and are driven by the embedded electronics. LED colors and blinking patterns may indicate, for example, current stimulation mode (say, green for ready, and red for active stimulation), and response characteristics (for example, yellow for match to baseline, and blue for latency deviation).

In some embodiments, auditory feedback is generated through a miniature piezoelectric buzzer embedded in the handle of the smart probe. The auditory feedback is driven by the embedded electronics and may encode event detection (e.g., muscle activation).

Depending on clinical application, the smart probe may be physically configured in either monopolar or bipolar form, each optimized for distinct anatomical targeting and electrode geometry (a monopolar smart probe includes a single active electrode contact at the distal tip, while a bipolar smart probe includes two closely spaced electrode contacts), even though both monopolar and bipolar forms support dynamic or real-time functional switching. The smart probe enables the user to control stimulation parameters whereas the passive monopolar and bipolar probes require dependency on another user to adjust parameters using the IONM software engine 110.

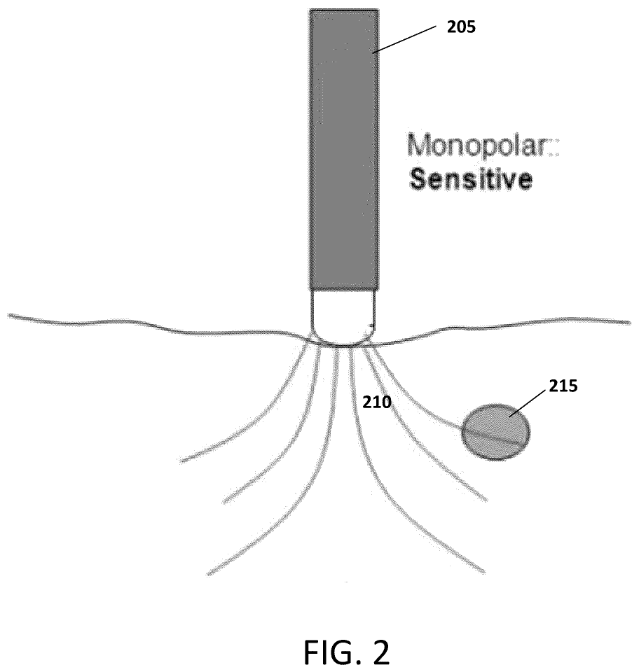

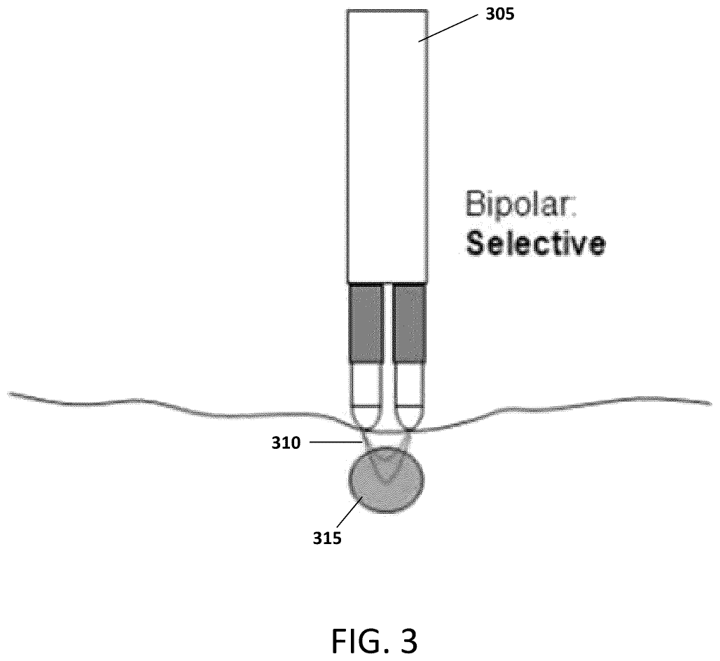

Referring back to FIG. 1A, in accordance with an aspect of the present specification, the stimulation module 120 is configured as a low current stimulator that supports any one or any combination of up to three stimulation modalities-such as, but not limited to, strip or grid electrode, monopolar probe and/or bipolar probe. Additionally, in some embodiments, the multi-modality stimulation module 190, that combines the stimulator functions with the 32-channel amplifier, also supports any one or any combination of up to three stimulation modalities-such as, but not limited to, strip or grid electrode, monopolar probe and/or bipolar probe. It should be appreciated that there may be scenarios where one or a combination of the three stimulation modalities may be of value in a surgical procedure, depending on a stage of a surgical procedure and/or based on what anatomical structure is being stimulated. For example, as shown in FIG. 2, a monopolar probe 205 is desirable when sensitivity of a physiological response from the nervous system (such as a muscle action potential) is of priority. The monopolar probe 205 has a spread or expanded field 210 of stimulation to ensure a stimulus encompasses a nerve structure 215. A discrete reference electrode is typically placed some distance away from where the monopolar stimulation probe 205 makes contact. As shown in FIG. 3, a bipolar probe 305 is desirable when selectivity of the stimulus is to be prioritized. The bipolar probe 305 has a focused field 310 of stimulation to apply stimuli to a selective nerve structure 315. However, a strip electrode may perform similarly to a monopolar or bipolar probe in terms of prioritization of sensitivity or selectivity, depending on which contacts of the strip electrode are activated.

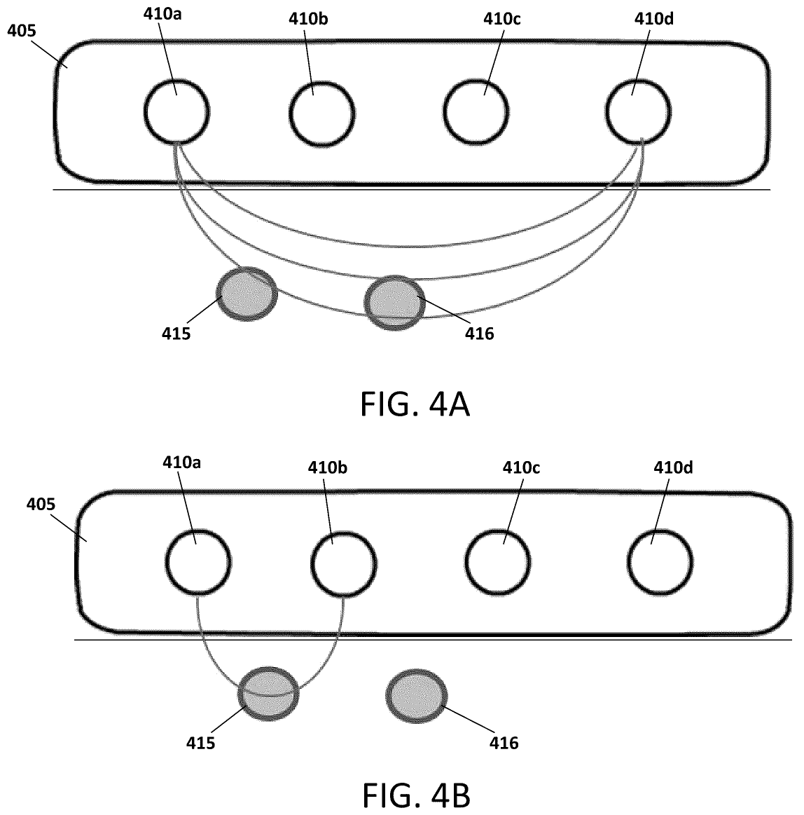

FIGS. 4A and 4B illustrate a strip electrode 405 comprising four electrical contacts 410a-410d, in accordance with an embodiment. In FIG. 4A, the strip electrode 405 is configured in a monopolar setup where two contacts 410a and 410d, that are away from each other, are activated. This setup results in stimulation of two neurological structures 415, 416 simultaneously. On the other hand, in FIG. 4B, the strip electrode 405 is configured in a bipolar setup where two contacts 410a and 410b, that are placed closer to each other, are activated. This setup results in stimulation of only one neurological structure 415.

Because an optimal stimulation paradigm may differ across patients and surgical procedure types, the multi-modality stimulation module 120 allows the user to easily prepare a varied neuro-stimulation setup, without having to physically move electrodes and/or probes and/or adjust the stimulus paradigm via dials and switches on a device at the computing device or near the OR (operating room) table.

### Stimulation Parameters, Protocols or Schedules

The IONM software application of the present specification implements a plurality of stimulation protocols or schedules, comprising a plurality of stimulation parameters, that are available to the user for automatic delivery or application to a patient depending at least upon a combination of the stimulation modalities configured at the stimulation module 120 of FIG. 1B, FIG. 1C, a neurostimulation and neuromonitoring objective such as, but not limited to, cortical stimulation or direct nerve stimulation and/or a surgical procedure being performed. It should be appreciated that the IONM software application provides the user with a degree of independence and automation with respect to delivery of stimuli and recordation of the stimuli as well as that of the correspondingly elicited neuromusculature response.

In various embodiments, stimulation protocols or schedules comprise driving a plurality of stimulation parameters such as, but not limited to, duration of the stimulation; time or moment of application of the stimulation sessions; intensity of stimulations, stimulation pulse shape, frequency, width and amplitude; stimulation duty cycle; stimulation continuity profile. Following are exemplary standard setting ranges for some of the stimulation parameters, including for motor cortex, speech, and language stimulation protocols and a direct nerve stimulation protocol: Pulse Width: 50 μsec to 2000 μsec (that is, 1000 μsec for single phase pulses and 2000 μsec for biphasic pulses since the positive and negative phases would each be 1000 μsec) and any increment therein.
Pulse Amplitude: 0.01 mA to 20 mA and any increment therein
Pulse Frequency: for a single pulse mode of stimulation, 0.5 Hz to 60 Hz (and any increment therein) for the multi-modality stimulation module 120 and 0.5 Hz to 130 Hz (and any increment therein) for the multi-modality stimulation module 190. However, when operating in pulse-train mode, the stimulation modules can deliver a series of pulses (“train”) multiple times per second, such that each train may itself contain several closely spaced pulses. In the pulse-train mode, in some embodiments, the train repetition rate (i.e., how many pulse trains are delivered per second) can be as high as 130 trains per second, while the intra-train pulse frequency (i.e., the rate of pulses within each train) can reach up to approximately 20,000 Hz. For example, the system can deliver 130 pulse trains per second, where each train contains pulses of 50 microseconds width separated by 0.5 microseconds, resulting in an intra-train pulse frequency of roughly 19,802 Hz.
Pulse Shape: Monophasic positive, monophasic negative, biphasic
Single Pulse Mode of Stimulation (i.e., direct nerve stimulation protocol), for the multi-modality stimulation module 120, comprising a single pulse stimulation, wherein the single pulse has a frequency in a range of 0.5 to 60 Hz (or any increment therein), a pulse width in a range of 50 μsec to 1000 μsec (or any increment therein), an inter-stimulus interval of 0.5 to 10 milliseconds (or any increment therein) and a pulse amplitude in a range of 0.01 mA to 20 mA for cranial nerves and a pulse amplitude in a range of 0.1 mA to 20 mA for peripheral nerves.
Single Pulse Mode of Stimulation (i.e., direct nerve stimulation protocol), in embodiments where the multi-modality stimulation module 190 combines the stimulator functions with the 32-channel amplifier, comprising a single pulse stimulation, wherein the single pulse has a frequency in a range of 0.5 to 130 Hz (or any increment therein), a pulse width in a range of 50 μsec to 1000 μsec, an inter-stimulus interval of 0.5 to 10 milliseconds (or any increment therein) and a pulse amplitude in a range of 0.01 mA to 35 mA or 0.01V to 20V for cranial nerves and a pulse amplitude in a range of 0.1 mA to 35 mA for peripheral nerves.
Constant-Voltage or Current Mode of Stimulation, in some embodiments, the multi-modality stimulation module 190 is capable of delivering stimulation pulses in constant-voltage mode, in addition to its constant-current capability. In the constant-voltage mode, the stimulator regulates its output so that the voltage across the connected electrodes remains fixed at a user-defined value—within a programmable range of 0.01 volts to 20 volts, while the pulse characteristics including frequency, pulse width, duration, and timing synchronization are identical to those used in constant-current stimulation mode. In the constant-current mode, the stimulator regulates its output so that the current across the connected electrodes remains fixed at a user-defined value—within a programmable range of 0.01 mA to 35 mA.
Multi-pulse train (MPT) stimulation (i.e. motor cortex stimulation protocol) comprising, for example, 1 to 10 pulses (or any increment therein), where each of the pulses is defined by a pulse width in a range of 50 μsec to 1000 μsec (or any increment therein), an inter-stimulus interval of 0.5 to 10 milliseconds (or any increment therein) and a pulse amplitude of 0.01 mA to 35 mA or 0.01V to 20V.
Duration of stimulation: 5 to 7 seconds

In various embodiments, the IONM software application implements a plurality of sub-sets of the aforementioned stimulation parameters and protocols depending at least upon the type of neurostimulation being delivered-such as, but not limited to, cortical stimulation or direct nerve stimulation.

Exemplary specifications of the multi-modality stimulation module 190 (characterized by a 32 channel amplifier, low current stimulator (LCS) and a software-controlled electrical stimulator switch matrix) are described below.

In some embodiments, the multi-modality stimulation module 190 includes several integrated functional and usability features that enhance its performance and adaptability in surgical and neurophysiological applications. The module 190 incorporates integrated electrosurgical unit (ESU) detection for automatic artifact management during electrocautery use, along with self-test capabilities that allow verification of system integrity and functionality prior to and during operation. The module 190 further includes a disposable overlay that enables custom input labeling to accommodate varied electrode configurations or procedural requirements. A status indicator is also provided to visually convey operational state, connection integrity, and system readiness. In some embodiments, the module 190 is optimized for direct cortical and subcortical mapping, providing precise stimulation and recording functionality suitable for intraoperative neurophysiological monitoring and brain mapping procedures.

32 channel amplifier characteristics, in accordance with some embodiments:
Total Inputs—33 inputs (32 referential channels with software selectable reference and 1 independent system reference input)
Noise (0.3-100 Hz)—less than 1 μVRMS
Input Impedance—100 MΩ
Sampling Rate—8 kHz
Mains Rejection Ratio—greater than 110 dB
A/D Resolution—24-bit
Notch Filter—50 or 60 Hz
Anti-aliasing Filter—greater than 40 dB
Maximum Input Range—35 mVₚₚ (depending on gain)
Frequency Range and Bandwidth—0.3 Hz to 2 kHz
Impedance—measurement of all inputs from 2 to 50 kΩ
Offset Voltage Allowed—+0.3 V

Low Current Stimulator (LCS) characteristics, in accordance with some embodiments:
Total Outputs—2 output pairs
Output Control Mode—constant current or constant voltage
Stimulus Polarity—Positive, negative, or alternating
Voltage Range—0.01 to 20 V (0.01 V steps)
Current Range—0.01 to 35 mA (0.01 mA steps)
Pulse Duration—50 to 1000 μs
Maximum Electrical Output Energy—1.225 mJ/pulse into 1 kΩ impedance
Repetition Rate—Up to 130 Hz (depending on sweep speed and interleave setup)
Pulse Train Limits—Up to 10 pulses
Interstimulus Interval—0.5 to 10 ms

### Use Case Illustrations

In accordance with various aspects of the present specification, the IONM system of the present specification enables the user to apply a plurality of stimulation protocols, patterns or schedules to the patient using at least one or any combination of the three stimulation modalities of the stimulation module with none and/or minimal physical or electromechanical intervention, monitoring and management from the user.

The IONM system of the present specification has application in a plurality of neurostimulation and neuromonitoring scenarios such as, but not limited to, cortical stimulation whereby the motor cortex is stimulated using a strip and/or probe(s) to determine functionality of the cortical structure(s) and direct nerve stimulation whereby a structure is stimulated to determine proximity to nervous system structures and wherein use of one or more types of stimulation probes may be advantageous to create stimulation fields of varying size/depth.

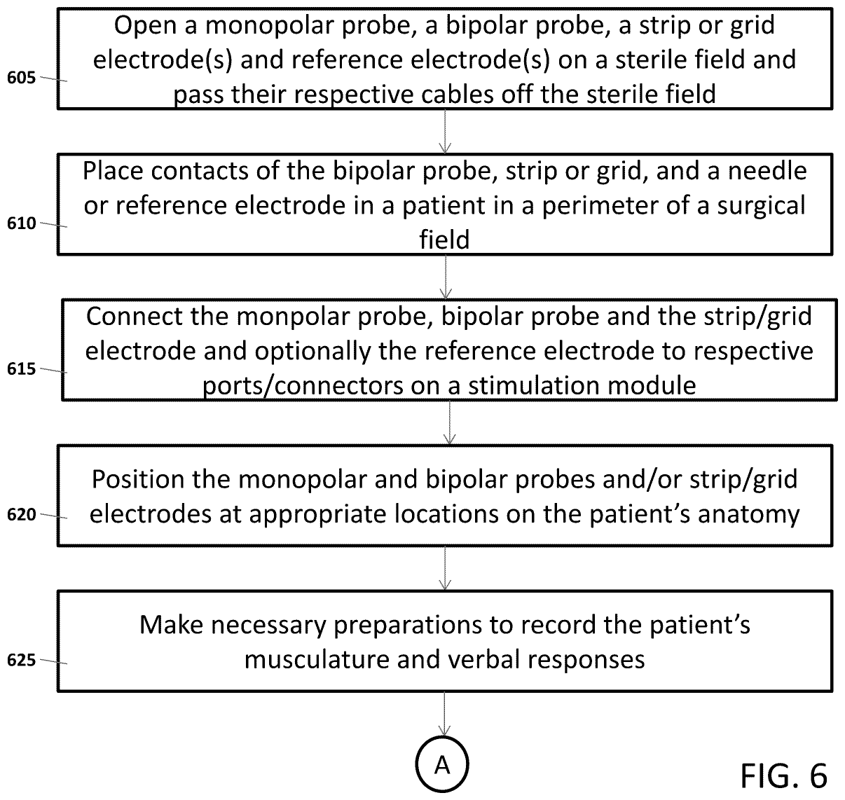

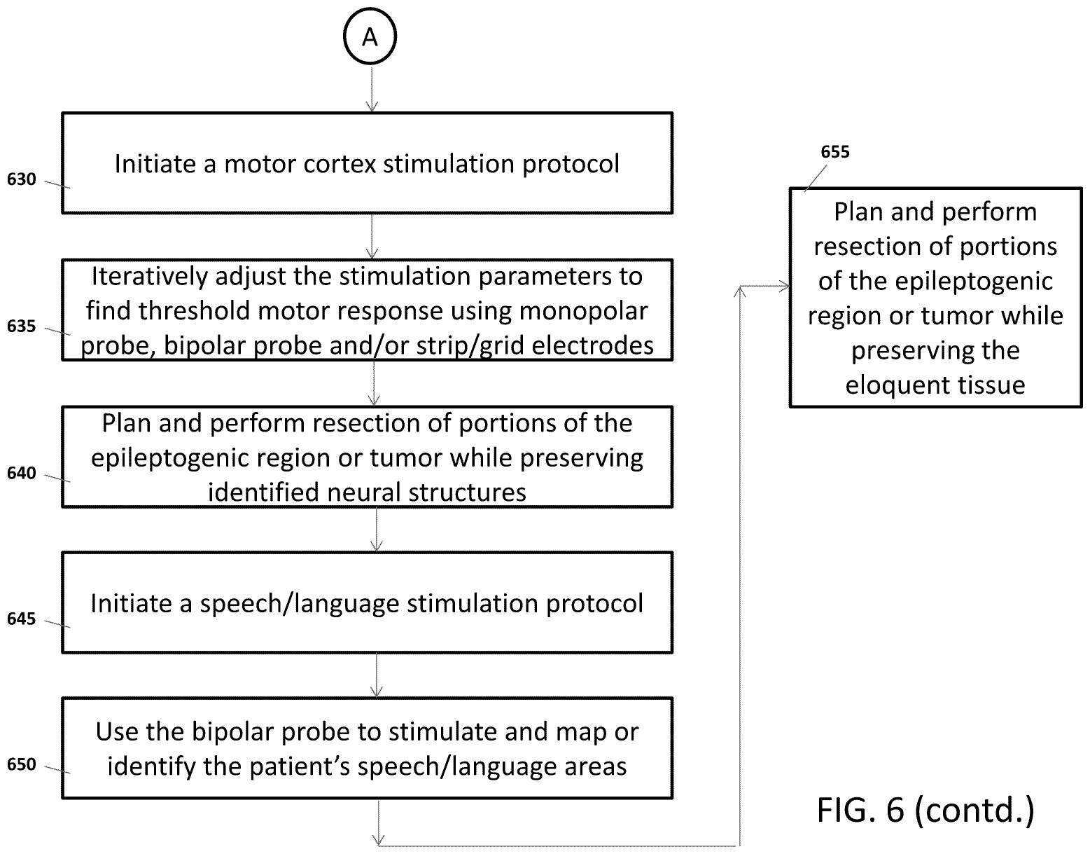

FIG. 6 is a flowchart illustrating a plurality of steps of a use case of cortical stimulation, using the IONM system of the present specification. Persons of ordinary skill in the art would appreciate that cortical stimulation may be employed in surgical procedures such as, but not limited to, craniotomy for resection of epileptogenic region and for resection of tumor. It should be appreciated that the steps of the use case of FIG. 6 are similar for craniotomy of either resection of epileptogenic region or tumor and that the procedures differ only in the number of strip and/or grid output channels or ports (of the stimulation module 120 of FIG. 1B or the stimulation module 190 of FIG. 1D) used.

Referring now to FIGS. 1B, 1D and 6, at step 605 a monopolar probe, a bipolar probe, a strip electrode, and/or grid electrode are opened on a sterile field and their respective connection cables are passed off the sterile field. Optionally, if a monopolar probe is being used, a reference electrode is also opened and its cable is passed off the sterile field. At step 610, contacts of the bipolar probe, monopolar probe, strip electrode and/or grid electrode, and a needle or a reference electrode (for a monopolar probe), are placed in the patient in a perimeter of a surgical field. At step 615, a user connects the monopolar and bipolar probes to the pair of probe ports 165a, 165b and the strip/grid electrode and optionally the reference electrode to the twelve connectors 160a-1601 of the stimulation module 120. Alternatively, at step 615, a user connects the monopolar and bipolar probes to the first and second pluralities of probe ports 194a, 194b and the strip/grid electrode and optionally the reference electrode to the thirty-two connectors of the stimulation module 190 that includes an integrated 32-channel amplifier.

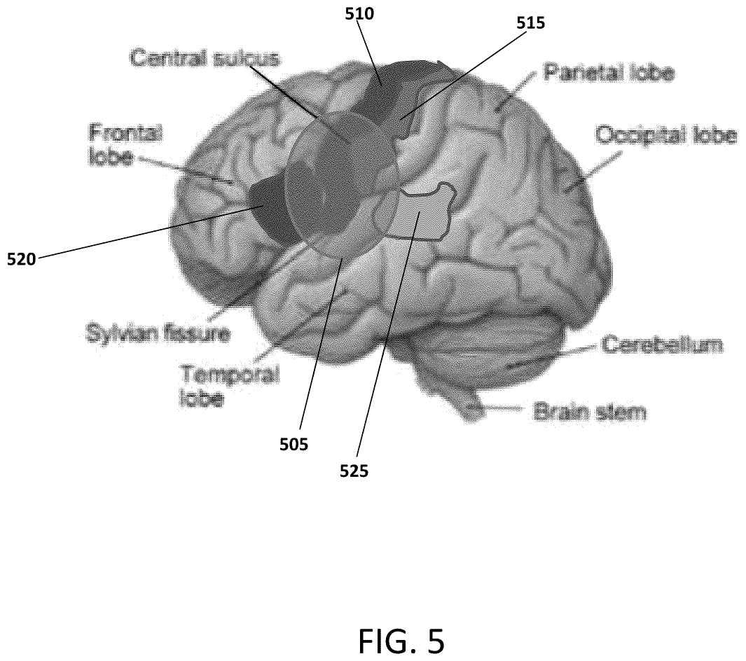

At step 620, the monopolar, bipolar probes, strip and/or grid electrodes are positioned at appropriate locations on the patient's anatomy to stimulate, identify, and assess functional areas related to motor, speech and language. In an embodiment, the monopolar probe is used for motor cortex stimulation and the bipolar probe is used for motor cortex and/or speech/language stimulation. FIG. 5 shows a tumor or epileptogenic region with reference to a map of the functional areas of a human brain, in accordance with an embodiment of the present specification. The figure shows the tumor or epileptogenic region 505 encompassing portions of the motor, sensory, speech and language areas 510, 515, 520, and 525. Consequently, resection of the tumor or epileptogenic region 505 poses significant risks to functions associated with the areas 510, 515, 520, and 525. To monitor integrity of neural structures associated with the areas 510, 515, 520, and 525, the monopolar probe is positioned in the motor cortex area 510 while the bipolar probe is sequentially positioned in the motor cortex 510 and/or speech, language areas 520, 525.

Referring back to FIG. 6, at step 625, necessary preparations are made to enable recordation of the patient's musculature responses as a result of motor cortex stimulation and of the patient's verbal responses as a result of stimulation of the speech/language areas.

In some embodiments, a plurality of recording or sensing electrodes are positioned at a plurality of muscle sites of the patient to record responses due to neurostimulation of the patient's motor cortex area. In an embodiment, the recording or sensing electrodes comprise pairs of EMG needle electrodes 125 of FIG. 1A placed in, for example, muscles of the face, arm and leg contralateral to the side of surgery. Recording muscles are chosen based on the location of the tumor or epileptogenic region. Example muscles include orbicularis oculi, orbicularis oris, masseter, mentalis, deltoid, biceps, flexor carpi ulnaris, flexor carpi radialis, abductor digiti minimi, adductor vastus lateralis, tibialis anterior, adductor hallucis. Also, responses to neurostimulation of speech/language areas are documented based on the patient's verbal responses.

At step 630, the user initiates a motor cortex stimulation protocol, using a graphical user interface of the IONM software application. It should be appreciated that the motor cortex stimulation protocol is one of a plurality of stimulation protocols pre-stored in a database associated with the IONM system. In some embodiments, the motor cortex stimulation protocol comprises of the following exemplary parameters and values/ranges:
Mode of Stimulation: Multi-pulse train (for example, 3-5 pulses)
Trigger: Single trigger (that is, single pulse stimulation)
Pulse Width: 500 μsec
Inter-stimulus interval: 2 to 4 milliseconds (equivalent to 250-500 Hz)
Pulse Amplitude: Up to 10 mA (permitted up to 20 mA)

In some embodiments, where the multi-modality stimulation module 190 (shown in FIG. 1D) combines the stimulator functions with a 32-channel amplifier, the motor cortex stimulation protocol comprises of the following exemplary parameters and values/ranges:
Mode of Stimulation: Multi-pulse train (for example, 3-5 pulses)
Trigger: Single trigger (that is, single pulse stimulation)
Pulse Width: 500 μsec
Inter-stimulus interval: 2 to 4 milliseconds (equivalent to 250-500 Hz)
Pulse Amplitude: Up to 10 mA (permitted up to 35 mA) or up to 20V

At step 635, the user iteratively adjusts the stimulation parameters, using at least one graphical user interface generated by the IONM software application or engine, to find threshold motor response using monopolar probe, bipolar probe and/or strip/grid electrodes and consequently identify or map functional areas of the motor cortex that need to be preserved during resection. The probes and/or strip/grid electrodes are utilized depending on whether sensitivity or specificity of stimulation is desired.

As an illustration, in one embodiment, the monopolar probe is applied to the patient's motor cortex area for stimulation and the pulse amplitude is modulated in a gradual stepped manner. In some embodiments, the pulse amplitude is modulated automatically by the IONM software engine. For example, the stimulation is initiated with 2 mA and stepped up, say by increments of 2 mA for example, to 10 mA till a muscle response is detected. Suppose that at 10 mA, a 200 μV EMG response is detected at deltoid, biceps, flexor carpi ulnaris. The pulse amplitude is now reduced to 9 mA and a 100 μV EMG response is detected at biceps, flexor carpi ulnaris. The pulse amplitude is now reduced to 8.5 mA that does not produce any EMG response from the muscles. Thus, the pulse amplitude of 9 mA is determined to be the threshold amplitude corresponding to the threshold EMG response. Consequently, the stimulated area is mapped or identified as corresponding to cerebral cortex representing biceps and flexor carpi ulnaris.

Now the bipolar probe is applied to the patient's motor cortex area for stimulation at the first threshold amplitude of 9 mA. However, in an embodiment, a stimulation at 9 mA using the bipolar probe may elicit a response only at the biceps. Consequently, the iterative stimulation process of determining the threshold amplitude and response (as done, earlier, using the monopolar probe) is repeated for the monopolar and bipolar probes at another site on the motor cortex. Let us assume that, at the other site, the threshold amplitude is determined to be 10 mA for orbicularis oculi and orbicularis oris using the monopolar probe and orbicularis oculi only using the bipolar probe.

Next, a strip electrode, such as the electrode 405 of FIGS. 4A, 4B with four contacts 410a-410d, and a reference electrode are placed over the motor cortex area identified as corresponding to orbicularis oculi, orbicularis oris, biceps and flexor carpi ulnaris (as already identified through the earlier performed iterative stimulation processes of determining the threshold amplitude and response). The IONM software now activates different combinations of the four contacts 410a-410d and a reference electrode to stimulate the identified motor cortex area to elicit corresponding motor response. For example, when contacts 410a, 410b are activated there may be no response, when contacts 410a, 410c are activated there may be a response from the orbicularis oculi only, when contact 410b and the reference electrode are activated there may be a response from the orbicularis oris only, when contact 410c and the reference electrode are activated there may be a response from the biceps only. However, when contacts 410b, 410d are activated there may be a response from all muscles. Accordingly, the combination of contacts 410b, 410d is determined to be ideal for frequent stimulation of the motor cortex during resection of the epileptogenic region or tumor.

At step 640, resection of portions of the epileptogenic region or tumor is planned and performed with intent to preserve the neural structures identified and associated with the identified contacts 410b, 410d which elicit response from all muscles. During resection, the monopolar probe is used to stimulate corticospinal tracts (CST) subcortically. An iterative stimulation process, such as one described above using the monopolar probe is used to estimate distance from CST. After completion of the monopolar probe stimulation, stimulation through the four contact strip electrode commences during the resection.

At step 645, the user initiates a speech/language stimulation protocol, using a graphical user interface of the IONM software application. It should be appreciated that the speech/language stimulation protocol is one of a plurality of stimulation protocols pre-stored in the database associated with the IONM system. In some embodiments, the speech/language stimulation protocol comprises of the following exemplary parameters and values/ranges:
Mode of Stimulation: Repetitive train stimulation.
Duration of stimulation: 5 to 7 seconds
Pulse Width: 200 μsec
Inter-stimulus interval: 16.6 milliseconds (equivalent to 60 Hz)
Pulse Amplitude: 5 mA (for speech/language responses, which are lower because a patient is awake)

At step 650, the bipolar probe is used to stimulate and map or identify the patient's speech/language areas. The patient is administered with speech/language tasks and the patient's verbal responses are documented while stimulation is delivered to tissues of the speech/language areas. Speech arrest and aphasia are examples of patient responses that are indicative that the stimulated tissue corresponds to speech/language functionality.

At step 655, resection of additional portions of the epileptogenic region or tumor is planned and performed with intent to preserve the identified eloquent tissue. Speech/language tasks continue to be administered to the patient throughout resection.

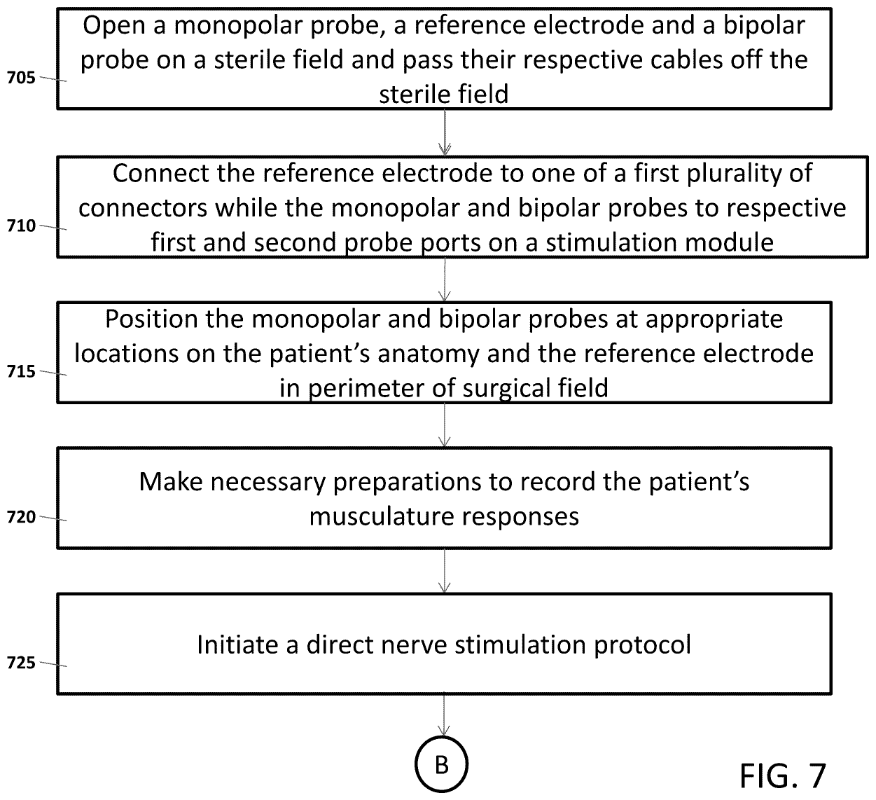

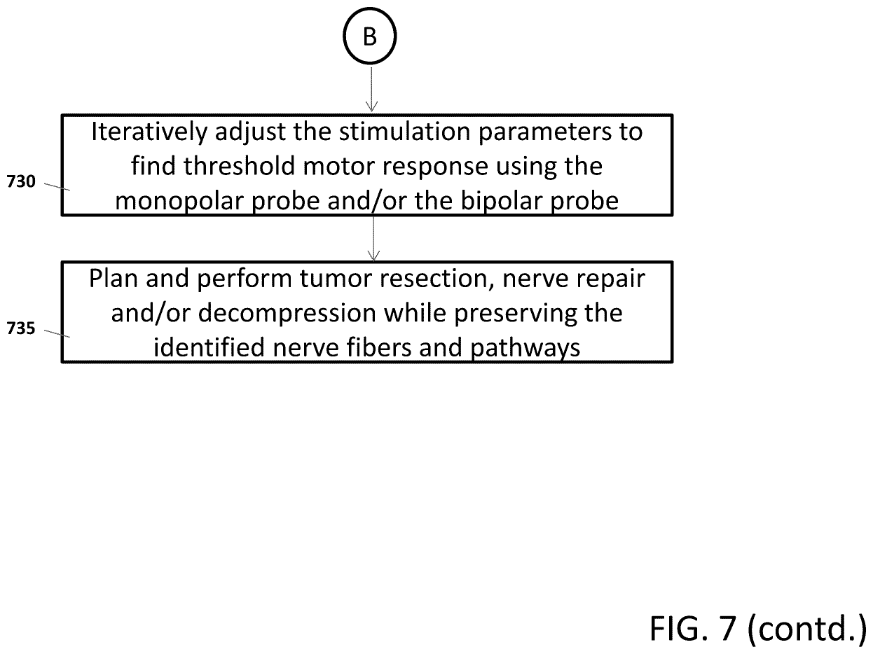

FIG. 7 is a flowchart illustrating a plurality of steps of another use case of direct nerve stimulation, using the IONM system of the present specification. Persons of ordinary skill in the art would appreciate that direct nerve stimulation may be employed in procedures such as, but not limited to, nerve graft, peripheral nerve tumor resection, brachial plexus repair, acoustic neuroma resection and microvascular decompression.

Referring now to FIGS. 1B, 1D and 7, at step 705 a monopolar probe, a reference electrode, and a bipolar probe are opened on a sterile field and their respective connection cables are passed off the sterile field. At step 710, a user connects the reference electrode to one of the first plurality of connectors 160 and the monopolar and bipolar probes to the pair of first and second probe ports 165a, 165b of the stimulation module 120. In some embodiments, the first plurality of connectors 160 is twelve. In some embodiments, as shown in FIG. 1D, the first plurality of connectors 192 is thirty-two.

At step 715, the monopolar and bipolar probes are positioned at appropriate target locations on the patient's anatomy for stimulation and the reference electrode is placed in a perimeter of the surgical field. In an embodiment, the target locations are associated with direct nerve stimulation of peripheral nerves comprising the brachial plexus. At step 720, necessary preparations are made to enable recordation of the patient's musculature responses as a result of direct nerve stimulation of peripheral nerves. In some embodiments, a plurality of recording or sensing electrodes are positioned at a plurality of muscle sites of the patient to record responses due to neurostimulation of the patient's peripheral nerves comprising the brachial plexus. In an embodiment, the recording or sensing electrodes comprise a plurality of pairs of EMG needle electrodes placed in, for example, muscles of the face, arm trunk and/or leg depending on the nerve(s) to be stimulated. Example muscles include trapezius, deltoid, biceps, triceps, flexor carpi ulnaris, flexor carpi radialis, abductor pollicis brevis, and abductor digiti minimi.

At step 725, the user initiates a direct nerve stimulation protocol, using a graphical user interface of the IONM software application. It should be appreciated that the direct nerve stimulation protocol is one of a plurality of stimulation protocols pre-stored in a database associated with the IONM system. In some embodiments, the direct nerve stimulation protocol comprises of the following exemplary parameters and values/ranges:
Mode of Stimulation: Single pulse stimulation
Frequency: 2 to 3 Hz
Pulse Width: 200 μsec
Inter-stimulus interval: 1 millisecond
Pulse Amplitude: Up to 2 mA for cranial nerves and 5 mA for peripheral nerves

At step 730, the user iteratively adjusts the stimulation parameters to find threshold response using monopolar probe and/or bipolar probe and consequently map or identify target nerve fibers and pathways that need to be preserved during resection or repair procedures. The probes are utilized depending on whether sensitivity or specificity of stimulation is desired.

As an illustration, in one embodiment, the monopolar probe is applied to the patient's target nerve bundle for stimulation and the pulse amplitude is modulated in a gradual stepped manner. In some embodiments, the pulse amplitude is modulated automatically by the IONM software engine. For example, the stimulation is initiated with 0.5 mA and stepped up, say by increments of 0.5 mA for example, to 2.5 mA till a muscle response is detected. Suppose that at 2.5 mA, a 200 μV EMG response is detected at deltoid, biceps, and triceps. The pulse amplitude is now reduced to 2.2 mA and a 100 μV EMG response is detected at biceps and triceps. The pulse amplitude is now further reduced to 2.1 mA that does not produce any EMG response from the muscles. Thus, the pulse amplitude of 2.2 mA is determined to be the threshold amplitude corresponding to the threshold EMG response of the nerve or nerve fibers that innervate the biceps and triceps. Consequently, the stimulated nerve or nerve fibers are mapped or identified as corresponding to cerebral cortex representing biceps and triceps.

Now the bipolar probe is applied to the patient's target nerve bundle for stimulation at the threshold amplitude of 2.2 mA. However, in an embodiment, the stimulation at 2.2 mA using the bipolar probe may elicit a response only at the biceps. Thus, the pulse amplitude of 2.2 mA is determined to be the threshold amplitude corresponding to the threshold EMG response of the nerve or nerve fibers that innervate the biceps. Consequently, the stimulated nerve or nerve fibers are mapped or identified as corresponding to cerebral cortex representing biceps.

Stimulation of the target nerve bundles is iteratively repeated at different sites of the patient's anatomy and musculature responses are recorded in order to map or identify nerve fibers and pathways that are critical to various motor functions and, therefore, need to be preserved.

At step 735, a procedure comprising tumor resection, nerve repair and/or decompression is planned and performed with intent to preserve the identified nerve fibers and pathways.

The above examples are merely illustrative of the many applications of the system and method of present specification. Although only a few embodiments of the present specification have been described herein, it should be understood that the present specification might be embodied in many other specific forms without departing from the spirit or scope of the specification. Therefore, the present examples and embodiments are to be considered as illustrative and not restrictive, and the specification may be modified within the scope of the appended claims.

We claim:

1. An intraoperative neurophysiological monitoring (IONM) system for using cortical stimulation to assess neural structures during a surgical procedure, the system comprising:
at least one reference electrode positioned in a perimeter of a surgical field of a patient;
at least one probe;
at least one strip electrode or grid electrode, wherein the at least one probe and/or the at least one strip electrode or grid electrode is positioned at target locations on the patient;
at least one sensing electrode positioned on said patient's and configured to record said patient's responses to stimulation;
a stimulation module, wherein the stimulation module is configured to:
function in a first mode of operation or a second mode of operation depending upon a type of the at least one probe, wherein the first mode of operation is a bipolar mode and the second mode of operation is a monopolar mode;
initiate a stimulation protocol in accordance with either the first mode of operation or second mode of operation;
adjust stimulation parameters of the stimulation protocol to determine a threshold response; and
use the at least one probe and/or the at least one strip electrode or grid electrode to determine whether the patient's neural structures are associated with at least one functional area based on the threshold response.

2. The IONM system of claim 1, wherein the stimulation protocol is a motor cortex stimulation protocol, a speech stimulation protocol, or a language stimulation protocol.

3. The IONM system of claim 1, wherein the stimulation module comprises a first plurality of output connectors and a second plurality of probe ports.

4. The IONM system of claim 3, wherein the first plurality of output connectors are configured to enable connection to the at least one strip electrode or grid electrode, wherein the at least one strip electrode or grid electrode comprises a plurality of contacts and wherein a total number of the plurality of contacts does not exceed a total number of the first plurality of output connectors.

5. The IONM system of claim 3, wherein the second plurality of probe ports comprises a first probe port and a second probe port, wherein each of the first probe port and the second probe port is configured to connect to the at least one probe, wherein the at least one probe comprises passive probes, and wherein the passive probes comprise at least one of a monopolar probe or a bipolar probe.

6. The IONM system of claim 3, wherein each of the first plurality of output connectors is configurable as either an anode or a cathode through a user interface in data communication with the IONM system.

7. The IONM system of claim 3, wherein each of the first plurality of output connectors is configurable as either an output for stimulation or an input for recording through a user interface in data communication with the IONM system.

8. The IONM system of claim 3, further comprising a computing device having a processor and a non-volatile memory for storing a plurality of programmatic instructions which, when executed, cause the processor to:
provide a user interface in data communication with the IONM system;
receive, via the user interface, user-defined stimuli, and
deliver signals representative of the user-defined stimuli to pairs of the first plurality of output connectors, wherein each of the plurality of output connectors is configurable as either an anode or a cathode through the user interface.

9. The IONM system of claim 3, wherein a number of the first plurality of output connectors is equal to thirty-two.

10. The IONM system of claim 1, wherein the at least one sensing electrode comprises an electromyography needle electrode.

11. The IONM system of claim 1, wherein the stimulation protocol comprises a multi-pulse train having 2 to 10 pulses and wherein each of the pulses is defined by a pulse width in a range of 50 μsec to 1000 μsec, an inter-stimulus interval in a range of 0.5 to 10 milliseconds and a pulse amplitude in a range of 0.01 mA to 35 mA or 0.01V to 20V.

12. An intraoperative neurophysiological monitoring (IONM) system adapted to use direct nerve stimulation to identify nerve fibers and nerve pathways during a surgical procedure, the system comprising:
at least one probe positioned at a first target location on the patient;
at least one sensing electrode positioned at a second target location in the patient; and
a stimulation module comprises a plurality of output connectors and a plurality of probe ports and is configured to:
function in a first mode of operation or a second mode of operation depending upon a type of the at least one probe, wherein the first mode of operation is a bipolar mode and the second mode of operation is a monopolar mode,
initiate a direct nerve stimulation protocol in accordance with either the first mode of operation or second mode of operation,
adjust stimulation parameters of the direct nerve stimulation protocol to determine a threshold motor response, and
use the at least one probe to identify certain of the patient's nerve fibers and nerve pathways based on the threshold motor response.

13. The IONM system of claim 12, wherein the at least one strip electrode or grid electrode has a total number of contacts not exceeding a total number of the plurality of output connectors, wherein each of the plurality of output connectors is adapted to connect to the at least one strip electrode or grid electrode.

14. The IONM system claim 12, wherein the plurality of probe ports comprises a first probe port and a second probe port and is configured to connect to the at least one probe, and wherein the at least one probe comprises at least one passive monopolar probe or passive bipolar probe.

15. The IONM system of claim 12, wherein each of the plurality of output connectors is configurable as either an anode or a cathode.

16. The IONM system of claim 12, wherein each of the plurality of output connectors is configurable as either an output for stimulation or an input for recording.

17. The IONM system of claim 12, further comprising a computing device having a processor and a non-volatile memory for storing a plurality of programmatic instructions which, when executed, cause the processor to:
provide a user interface in data communication with the IONM system;
receive, via the user interface, user-defined stimuli, and
deliver signals representative of the user-defined stimuli to pairs of the plurality of output connectors, wherein each of the plurality of output connectors is configurable as either an anode or a cathode through the user interface.

18. The IONM system of claim 12, wherein the at least one sensing electrode comprises an electromyography needle electrode.

19. The IONM system of claim 12, wherein the direct nerve stimulation protocol comprises a single pulse stimulation, and wherein the single pulse has a frequency of 0.5 Hz to 130 Hz, a pulse width of 50 μsec to 1000 μsec, and an interval between pulses of 0.5 millisecond to 10 milliseconds.

20. The IONM system of claim 19, wherein a pulse amplitude is in a range of 0.01 mA to 35 mA or 0.01V to 20V for cranial nerves and in a range of 0.1 mA to 35 mA for peripheral nerves.
